(19) United States
(12) Patent Application Publication
(10) Pub. No.: US 2025/0380944 A1
(43) Pub. Date: Dec. 18, 2025

(54) END TOOL AND SHAFT ASSEMBLY

(71) Applicant: LIVSMED INC., Seongnam-si (KR)

(72) Inventor: Woo Jung CHOI, Siheung-si (KR)

(21) Appl. No.: 19/237,895

(22) Filed: Jun. 13, 2025

(30) Foreign Application Priority Data
Jun. 14, 2024 (KR) 10-2024-0077426

(51) Int. Cl. A61B 17/072; A61B 17/00

(52) U.S. Cl. CPC A61B 17/072; A61B 2017/00314; A61B 2017/00323; A61B 2017/00477

(57) ABSTRACT

An end tool and shaft assembly includes an extension shaft defining a first longitudinal axis, and an end tool connected to one end portion of the extension shaft and configured to perform a joint motion about a first rotational axis perpendicular to the first longitudinal axis, the end tool defining a second longitudinal axis based on an extension direction of the end tool and configured to perform a second roll rotational motion using the second longitudinal axis as a rotational axis and a first roll rotational motion using the first longitudinal axis as a rotational axis.

### CROSS-REFERENCE TO RELATED APPLICATION

This application is based on and claims priority under 35 U.S.C. § 119 to Korean Patent Application No. 10-2024-0077426, filed on Jun. 14, 2024, in the Korean Intellectual Property Office, the disclosure of which is incorporated by reference herein in its entirety.

### BACKGROUND

### 1. Field

The present disclosure relates to a surgical instrument, and more particularly, to a surgical instrument that is mountable on a robot arm or operable manually, for use in laparoscopic surgery or various other surgical procedures.

### 2. Description of the Related Art

Medically, surgery refers to the treatment of diseases by cutting, slitting, or manipulating the skin, mucous membranes, or other tissues using medical devices In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

A surgical instrument is a tool equipped with an end tool provided on one end of a shaft that passes through a hole drilled in the skin, and is manipulated by a medical doctor by hand using a predetermined driving part or by a robot arm to perform surgery at the surgical site. The end tool provided on the surgical instrument performs a rotational motion, a gripping motion, a cutting motion, or the like through a predetermined structure.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure relates to an end tool and shaft assembly for a surgical instrument that is mountable on a robot arm or manually operable for use in laparoscopic surgery or various other surgical procedures, the end tool and shaft assembly minimizing wire usage and allowing the end tool to move in all directions.

In an embodiment of the present disclosure, an end tool and shaft assembly may include an extension shaft defining a first longitudinal axis, and an end tool connected to one end portion of the extension shaft and configured to perform a joint motion about a first rotational axis perpendicular to the first longitudinal axis, wherein the end tool defines a second longitudinal axis based on an extension direction of the end tool, and is configured to perform a second roll rotational motion using the second longitudinal axis as a rotational axis and a first roll rotational motion using the first longitudinal axis as a rotational axis.

In another embodiment of the present disclosure, the second longitudinal axis may be movable relative to the first longitudinal axis as the end tool performs the joint motion about the first rotational axis.

In the other embodiment of the present disclosure, the first roll rotational motion and the second roll rotational motion may be performed independently of each other.

In the other embodiment of the present disclosure, the end tool and shaft assembly may further include an internal shaft disposed inside the extension shaft and connected to the end tool, where the internal shaft may be configured to roll-rotate about the first longitudinal axis and may be roll-rotatable independently of the extension shaft.

In the other embodiment of the present disclosure, the end tool may further include a first hub connected to the extension shaft configured to be rotatable about the first rotational axis, and a link member at least partially disposed inside the first hub and configured to connect the internal shaft to the end tool.

In the other embodiment of the present disclosure, the link member may be configured to transmit a translational motion or a rotational motion of the internal shaft to the end tool.

In the other embodiment of the present disclosure, the end tool may be yaw-rotatable about the first rotational axis as the internal shaft moves forward or backward along the first longitudinal axis.

In the other embodiment of the present disclosure, the link member may be configured to transmit the translational motion or the rotational motion of the internal shaft to the end tool even when the end tool is in a yaw-rotated state.

In the other embodiment of the present disclosure, the link member may include a universal joint.

In the other embodiment of the present disclosure, the end tool may further include an end tool plate disposed on an end portion of the first hub at a distal end side and connected to the link member, wherein the end tool plate may be rotatable about the second longitudinal axis.

In the other embodiment of the present disclosure, the end tool may further include an operation-performing portion, which is coupled to the end tool plate and performs functions of a surgical instrument.

In the other embodiment of the present disclosure, the first hub may include a first coupling portion and a second coupling portion that extend toward the extension shaft and are configured to face each other, and the extension shaft may include a third coupling portion axially coupled to the first coupling portion and a fourth coupling portion axially coupled to the second coupling portion.

In the other embodiment of the present disclosure, the end tool may further include a first sub-shaft and a second sub-shaft positioned on the first rotational axis, wherein the first sub-shaft and the second sub-shaft are positioned to be spaced apart from each other by a certain degree, and the link member may be partially accommodated between the first sub-shaft and the second sub-shaft.

In the other embodiment of the present disclosure, the first sub-shaft may be axially coupled to the first coupling portion and the third coupling portion by passing therethrough, and the second sub-shaft may be axially coupled to the second coupling portion and the fourth coupling portion by passing therethrough.

In the other embodiment of the present disclosure, the end tool may further include a first coupling portion and a second coupling portion that extend toward the extension shaft and are configured to face each other, a first hub defining a second longitudinal axis, and a second hub having one end portion coupled to the extension shaft and another end portion connected to the first coupling portion and the second coupling portion of the first hub.

In the other embodiment of the present disclosure, the end tool may further include a yaw pulley rotatable about the first rotational axis and a yaw wire connected to the yaw pulley and configured to transmit power to the yaw pulley.

In the other embodiment of the present disclosure, the yaw pulley may be coupled to the first hub, and the end tool may be yaw rotatable in accordance with a rotation of the yaw pulley.

In the other embodiment of the present disclosure, the internal shaft may move forward or backward along the first longitudinal axis as an end tool yaw-rotates.

In the other embodiment of the present disclosure, the end tool may further include a jaw assembly coupled to a distal end of the end tool and including one or more jaws, and the link member may connect the internal shaft to the jaw assembly and be configured to transmit a rotational motion of the internal shaft to the jaw assembly.

In the other embodiment of the present disclosure, the end tool may further include a drive wire connected to the one or more jaws and configured to rotate the one or more jaws, the link member may include a through hole defined therein, and the drive wire may pass through the through hole and extend toward the extension shaft.

In the other embodiment of the present disclosure, the end tool may further include a guide tube configured to accommodate at least a portion of the drive wire therein and configured to be bendable to a predetermined degree, and the drive wire may be connected to the one or more jaws through an inside of the guide tube.

In the other embodiment of the present disclosure, the drive wire may be configured to be movable along the guide tube in the guide tube.

In the other embodiment of the present disclosure, the jaw assembly may include a first jaw including a staple cartridge and a second jaw including an anvil.

The present disclosure also relates to an end tool and shaft assembly including an extension shaft defining a first longitudinal axis and an end tool connected to one end portion of the extension shaft, and the end tool and shaft assembly may have a first roll degree of freedom defined by the first longitudinal axis, a joint motion degree of freedom of the end tool rotating about a first rotational axis different from the first longitudinal axis, and a second roll degree of freedom defined by a second longitudinal axis based on an extension direction of the end tool, and the first roll degree of freedom, the joint motion degree of freedom, and the second roll degree of freedom are each individually drivable.

The present disclosure also relates to a surgical instrument including an extension shaft defining a first longitudinal axis, an end tool connected to one end portion of the extension shaft and configured to perform a joint motion about a first rotational axis perpendicular to the first longitudinal axis, and a manipulation part connected to another end portion of the extension shaft and configured to manipulate an operation of the end tool, the end tool defining a second longitudinal axis based on an extension direction of the end tool, and configured to perform a second roll rotational motion using the second longitudinal axis as a rotational axis and a first roll rotational motion using the first longitudinal axis as a rotational axis.

In the other embodiment of the present disclosure, the end tool and shaft assembly may further include an internal shaft positioned inside the extension shaft and connected to the end tool, wherein the internal shaft may roll-rotate about the first longitudinal axis and may be roll-rotatable independently of the extension shaft.

In the other embodiment of the present disclosure, the surgical instrument may have a first roll degree of freedom defined by the first longitudinal axis, a joint motion degree of freedom of the end tool rotating about the first rotational axis different from the first longitudinal axis, and a second roll degree of freedom defined by a second longitudinal axis based on the extension direction of the end tool, the manipulation part may include a first driving part configured to drive the first roll degree of freedom, a second driving part configured to drive the joint motion degree of freedom of the end tool, and a third driving part configured to drive the second roll degree of freedom, and the first roll degree of freedom, the joint motion degree of freedom, and the second roll degree of freedom may each be individually driven.

In the other embodiment of the present disclosure, the first driving part may include a mechanism configured to roll-rotate an entirety of the second driving part and the third driving part.

In the other embodiment of the present disclosure, when the end tool roll-rotates about the second longitudinal axis, a positional relationship between the second longitudinal axis and the manipulation part remains unchanged.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a diagram for describing a first roll degree of freedom of an end tool and shaft assembly according to a first embodiment of the present disclosure.

FIG. 2 is a diagram for describing joint motion degrees of freedom of the end tool and shaft assembly according to the first embodiment of the present disclosure.

FIG. 3 is a diagram for describing a second roll degree of freedom of the end tool and shaft assembly according to the first embodiment of the present disclosure.

FIG. 4 is a diagram illustrating, together, respective rotational degrees of freedom of the end tool and the shaft assembly according to the first embodiment of the present disclosure.

FIG. 5 is a perspective view illustrating the end tool and shaft assembly according to the first embodiment of the present disclosure.

FIG. 6 is a view for describing an internal shaft of the end tool and shaft assembly of FIG. 5.

FIG. 7 is a perspective view illustrating the end tool and shaft assembly of FIG. 5 in a yaw-rotated state.

FIG. 8 is a perspective view illustrating the end tool and shaft assembly of FIG. 7 with a first hub and an extension shaft removed.

FIGS. 9 and 10 are perspective views illustrating the internal shaft of the end tool and shaft assembly of FIG. 7 in a roll-rotated state.

FIG. 11 is a perspective view illustrating the end tool and shaft assembly of FIG. 5 with the extension shaft removed.

FIG. 12 is a perspective view illustrating the internal shaft of the end tool and shaft assembly of FIG. 11 in a roll-rotated state.

FIGS. 13 and 14 are perspective views illustrating the end tool and shaft assembly of FIGS. 11 and 12 with the first hub and the extension shaft removed.

FIGS. 15 to 18 are perspective views illustrating the end tool rotating in response to a roll rotation of the extension shaft in the end tool and shaft assembly of FIG. 5.

FIG. 19 is a perspective view illustrating an end tool and shaft assembly according to a second embodiment of the present disclosure.

FIG. 20 is a magnified perspective view of the end tool and shaft assembly of FIG. 19 viewed from another angle.

FIG. 21 is a perspective view illustrating an end tool and shaft assembly according to a third embodiment of the present disclosure.

FIGS. 22 and 23 are views for describing a link member and a shaft of the end tool and shaft assembly of FIG. 21.

FIG. 24 is a perspective view illustrating an end tool and shaft assembly according to a fourth embodiment of the present disclosure.

FIG. 25 is a perspective view illustrating a surgical instrument to which the end tool and shaft assembly according to an embodiment of the present disclosure is applied.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become clear by referring to the detailed description below in conjunction with the accompanying drawings. However, the present embodiments are not limited to the embodiments disclosed below, but may be implemented in various forms.

In describing the present disclosure, a detailed description of known related arts will be omitted when it is determined that the essence of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

An end tool and shaft assembly according to an embodiment of the present disclosure is applicable to a surgical instrument, and more particularly, to a surgical instrument that is mountable on a robot arm or manually operable for use in laparoscopic surgery and various other surgical procedures.

The movement to be implemented in surgical tools required for the above-described laparoscopic surgery is to enable the end tool to rotate within a 180° hemisphere or a smaller angle of a spherical cone, while simultaneously rotating about its own axis. In other words, in the surgical instrument, the end tool needs to be movable vertically and horizontally relative to an extension shaft while also being roll-rotatable about its own axis. For example, surgical forceps consisting of a pair of jaws may perform an opening and closing motion vertically, or may perform a roll rotation to execute the opening and closing motion horizontally. This allows the surgical forceps to grasp body tissues located vertically or horizontally, while also enabling control over whether the forceps grasp the tissues in a vertical or horizontal direction.

In order to implement the above-described motions, conventional surgical tools configured the drive shafts in the order of yaw-pitch-roll or pitch-yaw-roll, and a roll motion was implemented by rotating the extension shaft.

When the end tool has three degrees of freedom—yaw, pitch, and roll—as described above, the manipulation of the surgical instrument can be intuitive, but the end tool requires separate yaw and pitch shafts. As a result, the size of a joint part of the end tool increases, which in turn leads to an increase in a rotation radius of the end tool.

Further, as described above, according to the related art, the surgical instrument requires a large number of pulleys to guide paths of wires used to drive the yaw and pitch shafts. Accordingly, as the wire passes through the large number of pulleys, the tension in the wire decreases due to friction, which in turn leads to a reduction in force used to operate the end tool.

To address the above-described issues, the end tool and shaft assembly according to an embodiment of the present disclosure aims to simplify the wire paths required to operate the end tool as much as possible. To this end, the end tool and shaft assembly according to an embodiment of the present disclosure may remove one degree of freedom from either yaw or pitch, and add one roll degree of freedom that rotates about the axis of the end tool.

That is, the present disclosure aims to provide an end tool and shaft assembly for a surgical instrument that minimizes wire usage and allows the end tool to move in all directions.

Hereinafter, rotational degrees of freedom of the end tool and shaft assembly according to an embodiment of the present disclosure will be described in detail with reference to the drawings.

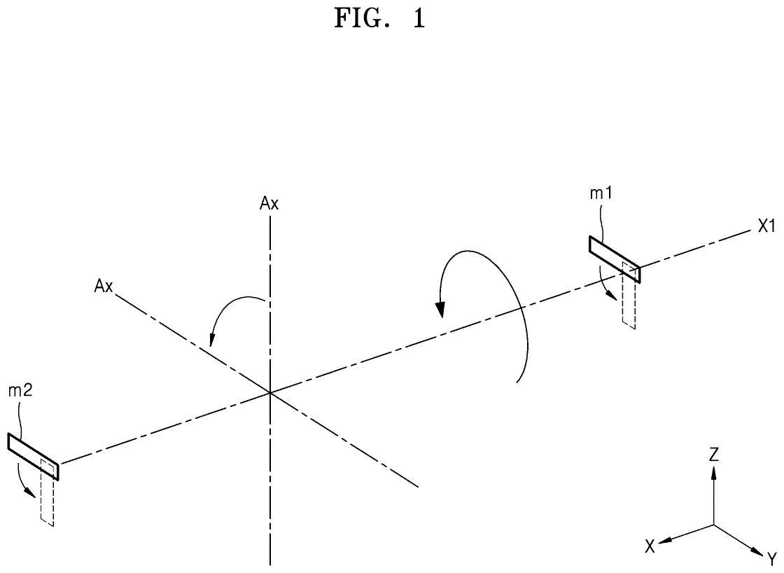

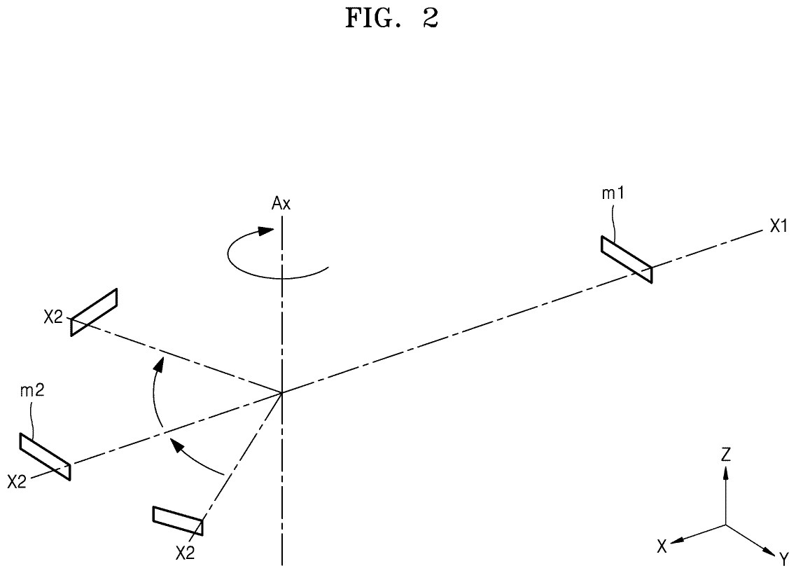

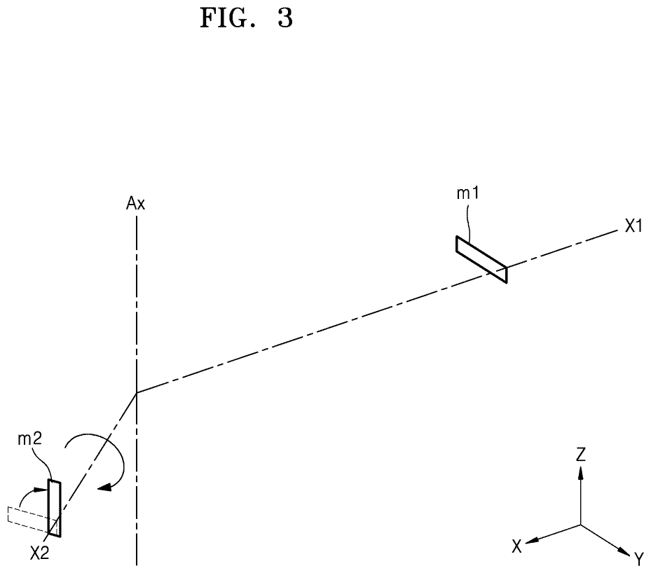

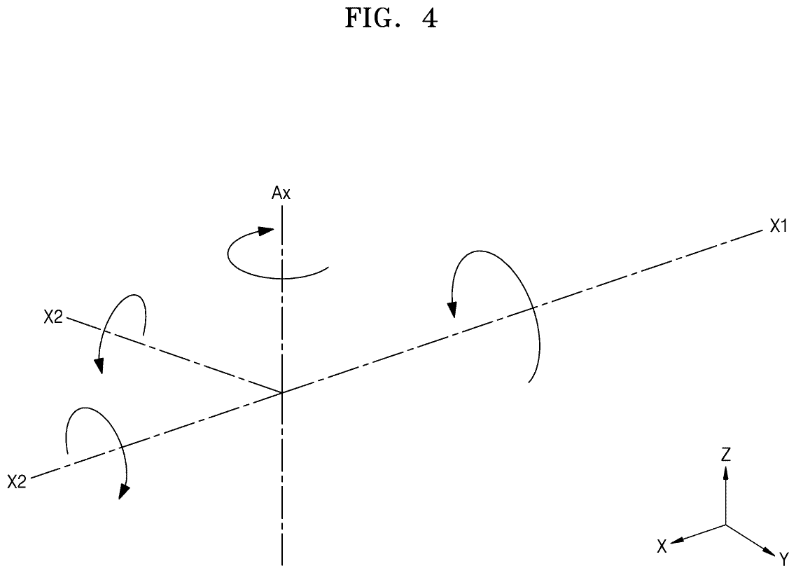

FIG. 1 is a diagram for describing a first roll degree of freedom of an end tool and shaft assembly according to a first embodiment of the present disclosure. FIG. 2 is a diagram for describing joint motion degrees of freedom of the end tool and shaft assembly according to the first embodiment of the present disclosure. FIG. 3 is a diagram for describing a second roll degree of freedom of the end tool and shaft assembly according to the first embodiment of the present disclosure. FIG. 4 is a diagram illustrating, together, respective rotational degrees of freedom of the end tool and the shaft assembly according to the first embodiment of the present disclosure.

FIGS. 1 to 4 are schematic diagrams that simply illustrate a first longitudinal axis, a second longitudinal axis, and a first rotational axis, while omitting the illustration of an end tool and an extension shaft according to the first embodiment of the present disclosure.

Here, X1 represents the first longitudinal axis, X2 represents the second longitudinal axis, and Ax represents the first rotational axis. In addition, m1 is a first marker indicating an axial rotation state of the first longitudinal axis X1, and m2 is a second marker indicating an axial rotation state of the second longitudinal axis X2.

Referring to FIGS. 1 to 4, the end tool and shaft assembly according to the first embodiment of the present disclosure may have the first roll degree of freedom, the joint motion degrees of freedom, and the second roll degree of freedom.

The first roll degree of freedom may be defined by the first longitudinal axis X1 of the extension shaft. In other words, the first roll degree of freedom may be described as a degree of freedom for a roll rotational motion of the extension shaft about the first longitudinal axis X1.

The second roll degree of freedom may be defined by the second longitudinal axis X2, which is based on an extension direction of the end tool. In other words, the second roll degree of freedom may be described as a degree of freedom for a roll rotational motion of the end tool about the second longitudinal axis X2.

The joint motion degree of freedom may be described as a degree of freedom for a joint motion of the end tool that rotates about the first rotational axis Ax, which is different from the first longitudinal axis X1.

As shown in FIG. 1, when the extension shaft roll-rotates about the first longitudinal axis X1, the first rotational axis Ax can rotate together with the extension shaft. That is, the first rotational axis Ax of the end tool can rotate about the first longitudinal axis X1 due to the roll rotation of the extension shaft. For example, when the extension shaft rotates 90° about the first longitudinal axis X1, the first rotational axis Ax, which was positioned on a Z-axis, may be positioned on a Y-axis.

Meanwhile, when the extension shaft roll-rotates about the first longitudinal axis X1, the end tool can also roll-rotate about the first longitudinal axis X1 together with the extension shaft. For example, when the first marker m1 rotates counterclockwise about the first longitudinal axis X1, the second marker m2 can also rotate counterclockwise together with the first marker m1.

Meanwhile, as shown in FIG. 2, when the end tool performs a joint motion about the first rotational axis Ax, the second longitudinal axis X2 can move relative to the first longitudinal axis X1. In other words, when the end tool performs a yaw rotation about the first rotational axis Ax, the second longitudinal axis X2 also performs a yaw rotation about the first rotational axis Ax, and an angle formed between the second longitudinal axis X2 and the first longitudinal axis X1 may change. For example, when the first rotational axis Ax is positioned on the Z-axis, the second longitudinal axis X2 and the second marker m2 may perform a yaw rotation on an XY plane.

Meanwhile, as shown in FIG. 3, the second roll degree of freedom allows the end tool to perform a roll rotation about the second longitudinal axis X2, even when in a yaw-rotated state. In other words, even when the second longitudinal axis X2 is not aligned with or parallel to the first longitudinal axis X1 but instead intersects with the first longitudinal axis X1, the end tool can still perform the roll rotational motion about the second longitudinal axis X2. That is, the second roll degree of freedom can be driven independently of the joint motion degree of freedom.

In addition, the second roll degree of freedom can be driven independently of the first roll degree of freedom. For example, when the end tool roll-rotates about the second longitudinal axis X2, a roll rotation of the extension shaft about the first longitudinal axis X1 may not be involved. In other words, the extension shaft may remain stationary, without performing a roll rotation, while the end tool alone roll-rotates about the second longitudinal axis X2. For example, the second marker m2 may rotate about the second longitudinal axis X2, while the first marker m1 remains stationary without rotating.

As such, the second roll degree of freedom, the first roll degree of freedom, and the joint motion degree of freedom may each be driven independently.

Hereinafter, the end tool and shaft assembly according to an embodiment of the present disclosure will be described in detail with reference to the drawings.

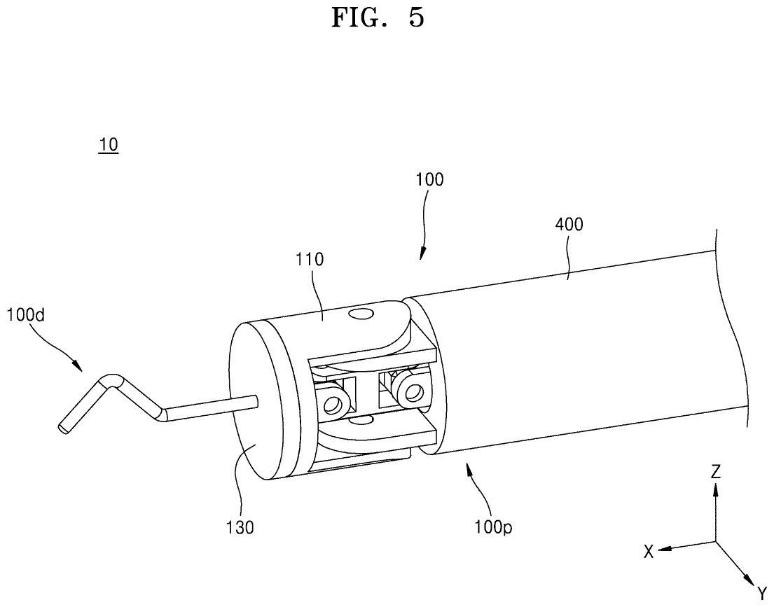

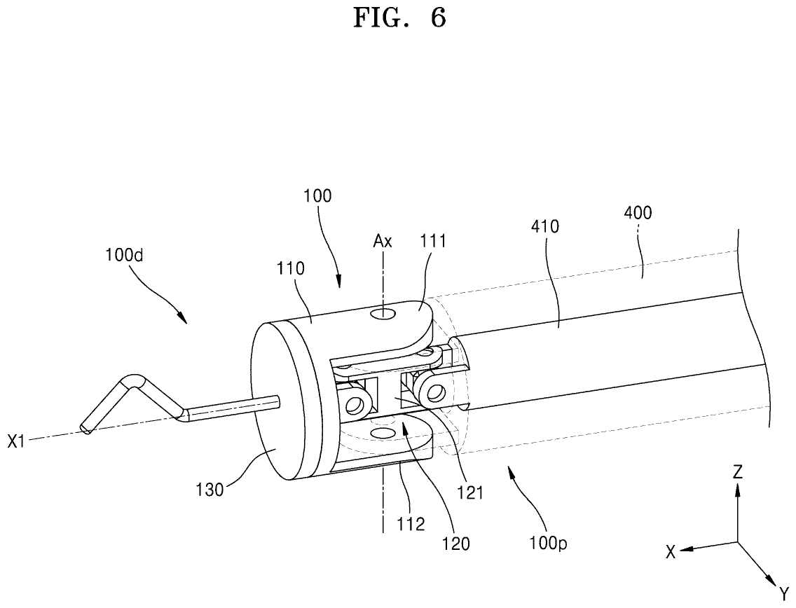

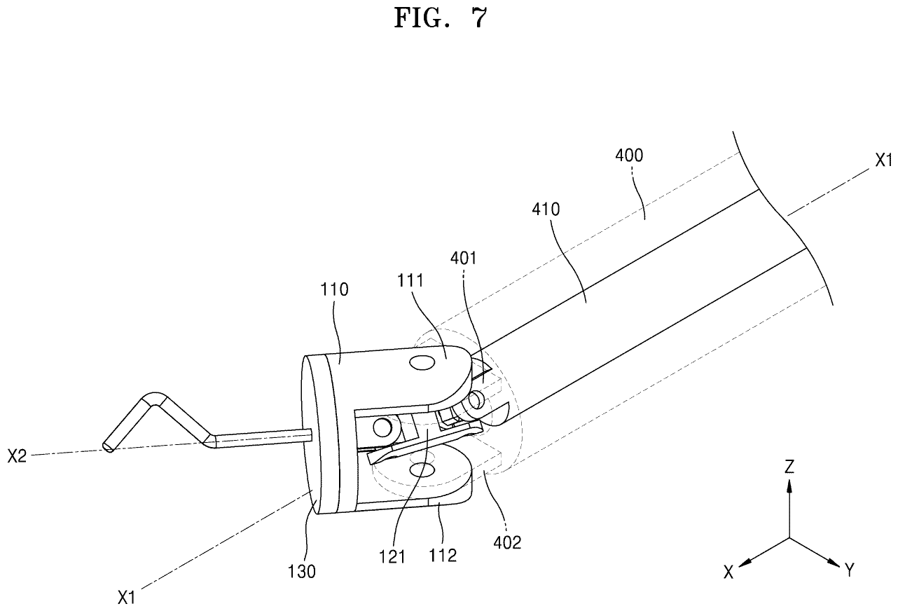

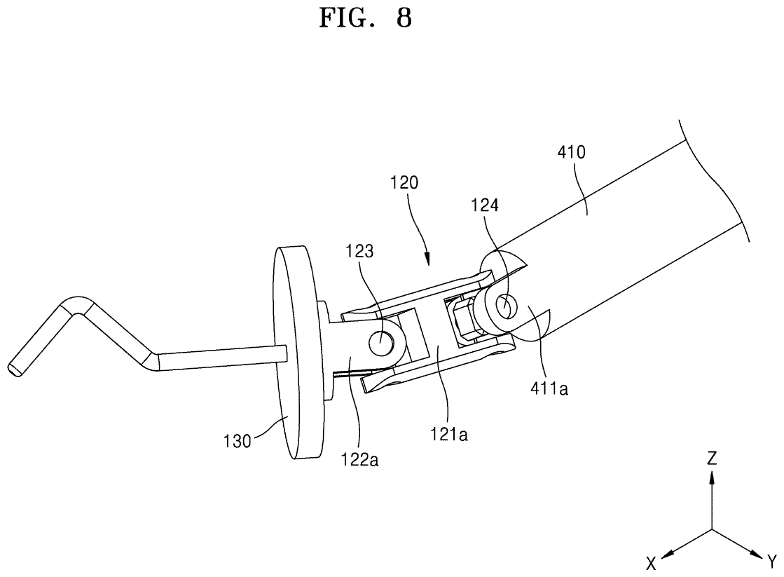

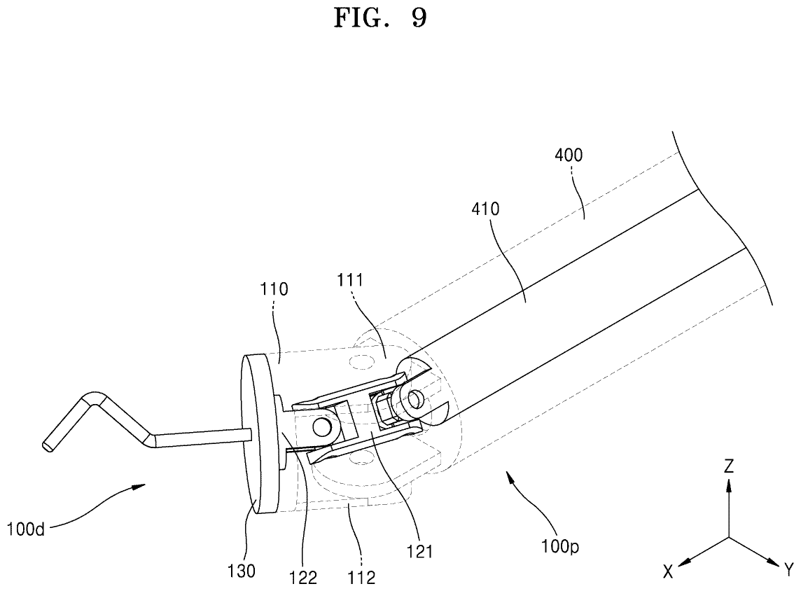

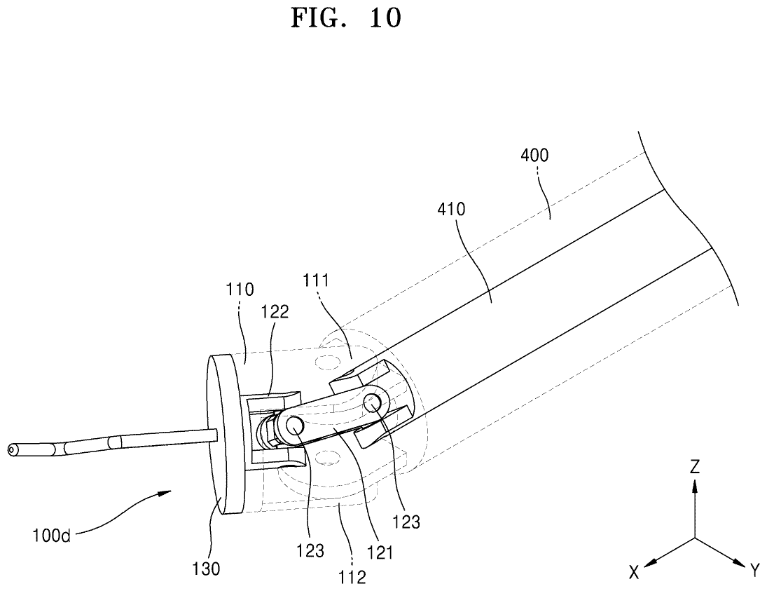

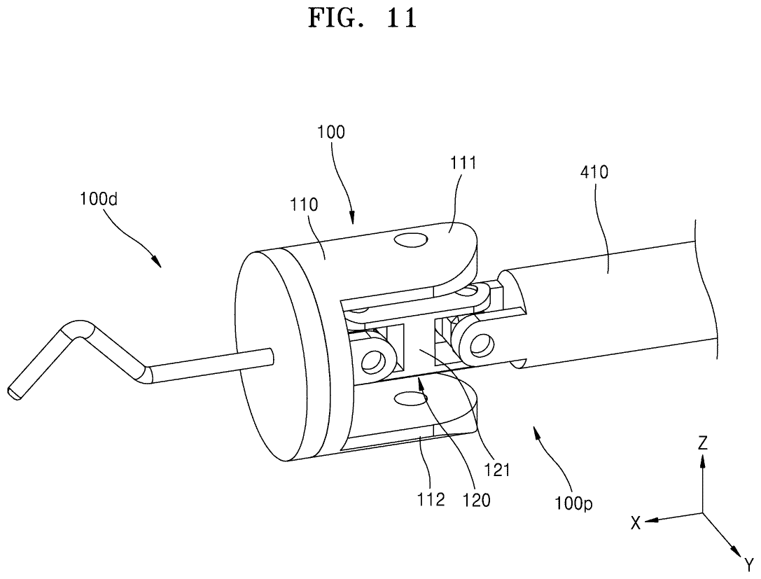

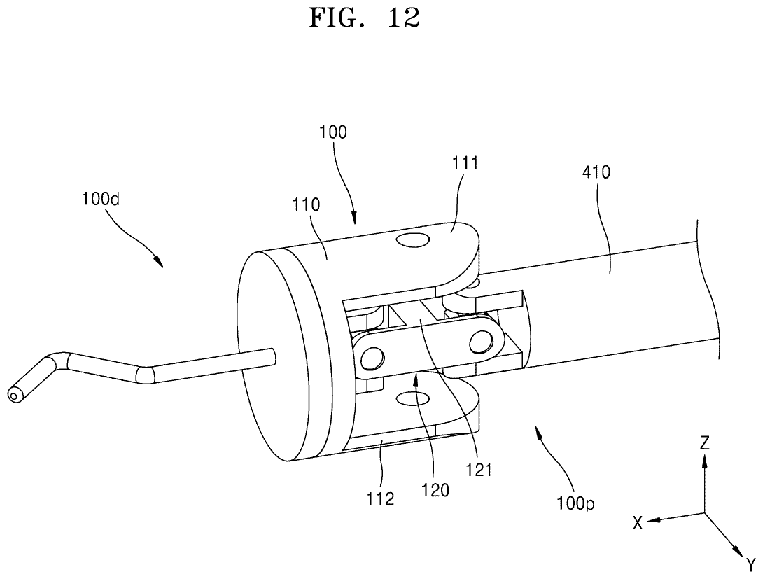

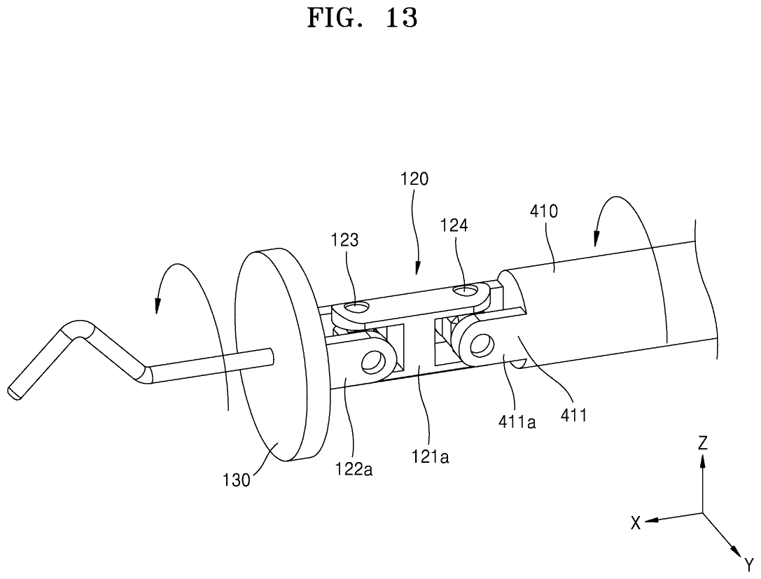

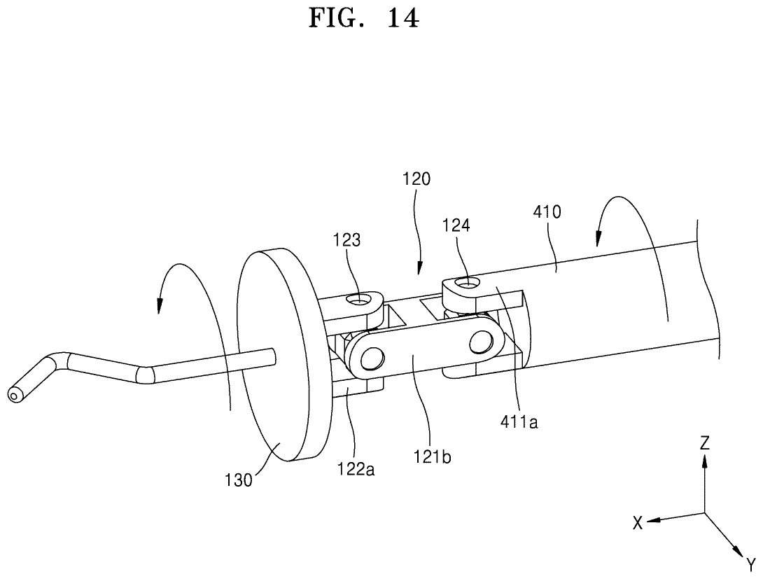

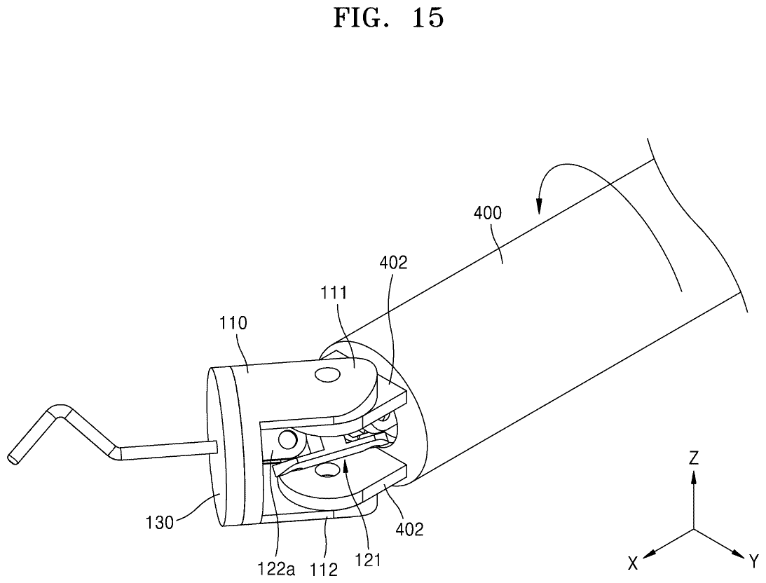

FIG. 5 is a perspective view illustrating the end tool and shaft assembly according to the first embodiment of the present disclosure, and FIG. 6 is a view for describing an internal shaft of the end tool and shaft assembly of FIG. 5. FIG. 7 is a perspective view illustrating the end tool and shaft assembly of FIG. 5 in a yaw-rotated state, and FIG. 8 is a perspective view illustrating the end tool and shaft assembly of FIG. 7 with a first hub and an extension shaft removed. FIGS. 9 and 10 are perspective views illustrating the internal shaft of the end tool and shaft assembly of FIG. 7 in a roll-rotated state. FIG. 11 is a perspective view illustrating the end tool and shaft assembly of FIG. 5 with the extension shaft removed, and FIG. 12 is a perspective view illustrating the internal shaft of the end tool and shaft assembly of FIG. 11 in a roll-rotated state. FIGS. 13 and 14 are perspective views illustrating the end tool and shaft assembly of FIGS. 11 and 12 with the first hub and the extension shaft removed, and FIGS. 15 to 18 are perspective views illustrating the end tool rotating in response to a roll rotation of the extension shaft in the end tool and shaft assembly of FIG. 5.

In describing the present disclosure, the portion closer to a user side, i.e., the portion closer to a manipulation part, will be referred to as a proximal end, and the portion farther from the user side, that is, the portion closer to an end portion of an end tool 100, will be referred to as a distal end.

For example, the portion of the end tool 100 closer to an extension shaft 400 will be defined and described as a proximal end 100p, and the portion of the end tool 100 farther from the extension shaft 400, that is, the portion near the end portion of the end tool 100, will be defined and described as a distal end 100d.

Referring to FIGS. 5 to 8, an end tool and shaft assembly 10 according to the first embodiment of the present disclosure may include the extension shaft 400, an internal shaft 410, and the end tool 100.

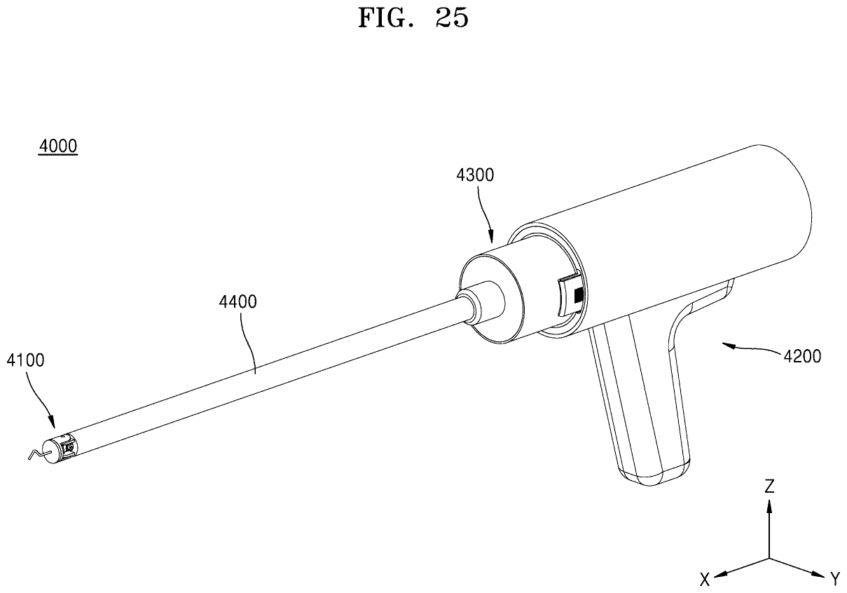

The extension shaft 400 defines the first longitudinal axis X1. The extension shaft 400 has one end portion connected to the end tool 100 and another end portion connected to a manipulation part (see 4200 in FIG. 25) of a surgical instrument, thereby serving to connect the manipulation part to the end tool 100. In addition, the extension shaft 400 is formed in a hollow shape, allowing one or more wires or cables to be accommodated therein.

Further, the extension shaft 400 may accommodate the internal shaft 410, which will be described later, therein. In addition, the extension shaft 400 and the internal shaft 410 may each be connected to the end tool 100.

Meanwhile, the extension shaft 400 may roll-rotate about the first longitudinal axis X1. In other words, a roll degree of freedom of the surgical instrument may also be implemented through the extension shaft 400. This will be described in detail later.

Meanwhile, the extension shaft 400 may include a coupling portion at one end portion thereof, which is connected to the end tool 100, to facilitate its coupling with the end tool 100.

The internal shaft 410 may be disposed inside the extension shaft 400. The internal shaft 410 may have one end portion connected to the end tool 100 and another end portion connected to the manipulation part. That is, the internal shaft 410 may transmit a driving force resulting from the operation of the manipulation part to the end tool 100.

Specifically, the internal shaft 410 can roll-rotate about the first longitudinal axis X1 and independently perform a roll rotation with respect to the extension shaft 400. In other words, the extension shaft 400 can remain stationary without performing a roll rotation while only the internal shaft 410 roll-rotates, or conversely, the extension shaft 400 can roll-rotate while the internal shaft 410 remains stationary without performing a roll rotation. Of course, the extension shaft 400 and the internal shaft 410 can also roll-rotate together.

Here, as the internal shaft 410 can independently roll-rotate with respect to the extension shaft 400, an additional roll degree of freedom can be provided. This will be described in detail later.

Meanwhile, the internal shaft may perform a translational motion by moving forward or backward along an axis of the extension shaft 400 while positioning inside the extension shaft 400. In other words, the internal shaft 410 may perform a roll rotation about the first longitudinal axis X1 while simultaneously performing the translational motion, and may also perform the roll rotation and the translational motion independently of each other.

The end tool 100 may be connected to one end portion of the extension shaft 400. In addition, the end tool 100 may be connected to one end portion of the internal shaft 410. Accordingly, the end tool 100 can receive the driving force resulting from the operation of the manipulation part and perform various motions.

Specifically, the end tool 100 may be connected to one end portion of the extension shaft 400 and one end portion of the internal shaft 410, thereby enabling roll rotational motion about the first longitudinal axis X1 and joint motion about the first rotational axis Ax different from the first longitudinal axis X1.

In other words, the end tool 100 can perform yaw rotation about the first rotational axis Ax and roll rotational motion about the first longitudinal axis X1.

The end tool 100 may perform necessary motions for surgery by being inserted into a surgical site. As an example of the end tool 100, as shown in FIG. 5 or the like, a hook may be used. However, the principle of the present disclosure is not limited thereto, and various devices for performing surgery may be used as the end tool 100. For example, as the end tool, a pair of jaws may be used, and surgical tools such as forceps, needle holders, dissectors, and staplers may also be used. In addition, as the end tool, surgical tools such as monopolar dissectors, monopolar scissors, monopolar hooks, monopolar spatulas, bipolar dissectors, bipolar forceps, and vessel sealers may be used for electrocautery.

The end tool 100 according to the first embodiment of the present disclosure may include a first hub 110, an end tool plate 130, and a link member 120.

The first hub 110 defines the second longitudinal axis X2. The first hub 110 may be rotatably connected to one end portion of the extension shaft 400. Here, the first hub 110 may include a coupling portion, which is a portion that connects the first hub 110 to the extension shaft 400. Specifically, the first hub 110 may include a first coupling portion 111 and a second coupling portion 112.

Here, the first coupling portion 111 and the second coupling portion 112 may be formed to extend toward the extension shaft 400 and to face each other.

In other words, the first coupling portion 111 and the second coupling portion 112 form outer side portions of the first hub 110, and may be formed parallel to each other. In addition, as the first coupling portion 111 and the second coupling portion 112 are formed to be spaced apart from each other, other components may be accommodated in a space therebetween. For example, the first hub 110 may accommodate the link member 120, which will be described later, in the space between the first coupling portion 111 and the second coupling portion 112.

The first coupling portion 111 and the second coupling portion 112 may be axially coupled to the coupling portion of the extension shaft 400 to connect the first hub 110 to the extension shaft 400. Specifically, the extension shaft 400 may include a third coupling portion 401 axially coupled to the first coupling portion 111 and a fourth coupling portion 402 axially coupled to the second coupling portion 112.

In other words, the first hub 110 and the extension shaft 400 may be axially coupled to each other to enable yaw rotation about the first rotational axis Ax.

Further, the end tool 100 may include a first sub-shaft (not shown) and a second sub-shaft (not shown) disposed along the first rotational axis Ax.

Here, the first sub-shaft and the second sub-shaft are disposed to be spaced apart to a certain extent, allowing a portion of the link member 120 to be accommodated between the first sub-shaft and the second sub-shaft.

In addition, the first sub-shaft may be axially coupled to the first coupling portion 111 and the third coupling portion 401 by passing therethrough, while the second sub-shaft may be axially coupled to the second coupling portion 112 and the fourth coupling portion 402 by passing therethrough.

Meanwhile, in a modified example of an embodiment of the present disclosure, the end tool 100 may further include a second hub (not shown) in addition to the first hub 110.

Here, the second hub may be a portion that connects the first hub 110 to the extension shaft 400. In FIG. 5 or the like, the coupling portions, which are axially coupled to the first coupling portion 111 and the second coupling portion 112 of the first hub 110, are illustrated as being formed on the extension shaft 400 to directly connect the extension shaft 400 to the first hub 110. However, the concept of the present disclosure is not limited thereto, and it is, of course, possible for other components, such as the second hub, to be interposed between the first hub 110 and the extension shaft 400 to connect the first hub 110 to the extension shaft 400.

In other words, one end portion of the second hub may be connected to the first hub 110, and another end portion thereof may be connected to the extension shaft 400. Specifically, the second hub may include coupling portions at one end portion thereof, which are similar to the third coupling portion 401 and the fourth coupling portion 402 of the extension shaft 400.

That is, the second hub may include a fifth coupling portion (not shown) corresponding to the third coupling portion 401 and a sixth coupling portion (not shown) corresponding to the fourth coupling portion 402.

Defining this from another perspective, the second hub may include the fifth coupling portion axially coupled to the first coupling portion 111, and the sixth coupling portion axially coupled to the second coupling portion 112.

In other words, the first hub 110 and the second hub may be axially coupled to each other to enable yaw rotation about the first rotational axis Ax.

In addition, the first sub-shaft may be axially coupled to the first coupling portion 111 and the fifth coupling portion by passing therethrough, while the second sub-shaft may be axially coupled to the second coupling portion 112 and the sixth coupling portion by passing therethrough.

The end tool plate 130 may be disposed on the distal end 100d of the end tool 100. Specifically, the end tool plate 130 may be disposed on an end portion of the first hub 110 at the distal end 100d side.

Here, the end tool plate 130 may be rotatable about the second longitudinal axis X2. In other words, the end tool plate 130 may be connected to the first hub 110 and be rotatable with respect thereto.

The end tool plate 130 may be connected to the link member 120, which will be described later, and may be connected to the internal shaft 410 via the link member 120. Specifically, one end portion of the end tool plate 130 may be connected to the link member 120, thereby allowing the end tool plate 130 to roll-rotate about the second longitudinal axis X2 due to the rotation of the link member 120.

The end tool plate 130 may include an operation-performing portion on the distal end 100d side. Here, the operation-performing portion is a portion that performs functions of a surgical instrument, and may be formed integrally with the end tool plate 130 or as a separate assembly that is coupled to the end tool plate 130.

In other words, the end tool 100 may include the operation-performing portion, which is coupled to the end tool plate 130 and performs the functions of a surgical instrument.

Here, the operation-performing portion may refer to any of the various devices of the end tool 100 described above. That is, the operation-performing portion may include a hook. However, the concept of the present disclosure is not limited thereto, and various devices for performing surgery may be used as the operation-performing portion. For example, as the operation-performing portion, a pair of jaws may be used, and surgical tools such as forceps, needle holders, dissectors, and staplers may also be used. In addition, as the operation-performing portion, surgical tools such as monopolar dissectors, monopolar scissors, monopolar hooks, monopolar spatulas, bipolar dissectors, bipolar forceps, and vessel sealers may be used for electrocautery.

At least a portion of the link member 120 may be disposed in the first hub 110. Alternatively, at least a portion of the link member 120 may be disposed in the first hub 110 or the second hub (not shown).

The link member 120 may connect the internal shaft 410 to the end tool 100 and transmit a translational motion or rotational motion of the internal shaft 410 to the end tool 100.

Specifically, the link member 120 may connect the internal shaft 410 to the end tool plate 130.

Accordingly, as the internal shaft 410 roll-rotates about the first longitudinal axis X1, the end tool plate 130 connected to the link member 120 may roll-rotate relative to the first hub 110. That is, only the internal shaft 410 and the end tool plate 130 connected thereto can roll-rotate, while the first hub 110 and the extension shaft 400 remain stationary without performing roll rotation about the first longitudinal axis X1. In other words, only the operation-performing portion can roll-rotate, while the first hub 110 of the end tool 100 remains stationary without performing roll rotation.

Referring to FIGS. 7 to 10, the link member 120 may transmit the rotational motion of the internal shaft 410 to the end tool 100, even when the end tool 100 is in a yaw-rotated state.

Here, a non-yaw-rotated state of the end tool 100 refers to a state in which the second longitudinal axis X2 is aligned with or parallel to the first longitudinal axis X1, while the yaw-rotated state refers to a state in which the second longitudinal axis X2 forms a certain angle with the first longitudinal axis X1.

In other words, the end tool and shaft assembly 10 according to an embodiment of the present disclosure can transmit a roll rotational motion of the internal shaft 410 to the end tool 100, even when the second longitudinal axis X2 of the end tool 100 is rotated at a certain angle relative to the first longitudinal axis X1.

As such, a degree of freedom for roll rotation of the internal shaft 410, the end tool plate 130, and the operation-performing portion, independent of roll rotation of the extension shaft 400 and the first hub 110, may be defined as the second roll degree of freedom.

In addition, a degree of freedom for roll rotation of the entire extension shaft 400 and end tool 100 may be defined as the first roll degree of freedom. In addition, a degree of freedom for the joint motion of the end tool 100 rotating about the first rotational axis may be defined as the joint motion degree of freedom or a yaw degree of freedom.

In other words, the end tool and shaft assembly 10 according to an embodiment of the present disclosure may be configured with two roll degrees of freedom and one yaw degree of freedom.

As described above, in order to implement the second roll degree of freedom, a mechanism is required to transmit the roll rotational motion of the internal shaft 410 to the end tool plate 130, even when the end tool 100 is in a yaw-rotated state. In other words, the mechanism should be able to transmit rotational force to the end tool plate 130 even when a rotational axis of the internal shaft 410, which roll-rotates about the first longitudinal axis X1, is different from a rotational axis of the end tool plate 130. The link member 120 for implementing such a mechanism may include a universal joint.

For example, the link member 120 may include a single cardan joint. Preferably, the link member 120 may include a double cardan joint, which can transmit rotational force even when the end tool 100 is yaw-rotated by 90° with respect to the first rotational axis Ax.

However, the concept of the present disclosure is not necessarily limited thereto, and any mechanism capable of transmitting the rotational force of the internal shaft 410 to the end tool 100, even when the end tool 100 is in a yaw-rotated state, may, of course, be used as the link member 120. For example, the link member 120 may include a flexible shaft or a flexible coupling.

Hereinafter, an embodiment in which a double cardan joint is used as the link member 120 will be described in detail.

The link member 120 may include a central body 121, and a first spider 123 and a second spider 124 that are axially coupled to both end portions of the central body 121, respectively. In addition, a first yoke 122 axially coupled to the first spider 123 may be formed on the end tool plate 130. Similarly, a second yoke 411 axially coupled to the second spider 124 may be formed on one end portion of the internal shaft 410.

The first yoke 122 may be formed to extend from the end tool plate 130 toward the proximal end 100p. The second yoke 411 may be formed to extend from one end portion of the internal shaft 410 toward the distal end 100d.

The first yoke 122 may include a pair of yoke portions 122a, which face each other and are parallel, and the second yoke 411 may also include a pair of yoke portions 411a that face each other and are parallel. The first yoke 122 and the second yoke 411 may be formed with the same size and shape, but may, of course, also be formed with different sizes and shapes.

The central body 121 may be formed with a length corresponding to a length of the first hub 110, the length allowing the end tool plate 130 and the internal shaft 410 to be connected to each other. The central body 121 may include an upper portion 121b, a lower portion 121b, and a central portion 121a connecting the upper portion 121b to the lower portion 121b.

Hereinafter, the roll rotation and yaw rotation of the end tool and shaft assembly 10, based on three degrees of freedom according to the first embodiment of the present disclosure, will be described in detail.

First, the second roll degree of freedom may be implemented through the internal shaft 410.

FIG. 12 illustrates a state in which the internal shaft 410 of FIG. 11 has roll-rotated counterclockwise by 90°. FIG. 14 illustrates a state in which the internal shaft 410 of FIG. 13 has roll-rotated counterclockwise by 90°.

Referring to FIGS. 11 to 14, the internal shaft 410 may be connected to the link member 120, and the link member 120 may be connected to the end tool plate 130. As described above, the end tool plate 130 may be a member capable of rotating relative to the first hub 110. Thus, when the internal shaft 410 roll-rotates about the first longitudinal axis X1, the link member 120 connected to the internal shaft 410 can transmit rotational force of the internal shaft 410 to the end tool plate 130. Accordingly, the end tool plate 130 can roll-rotate together with the internal shaft 410 in response to the rotation of the link member 120.

In addition, the end tool plate 130 can roll-rotate along with the internal shaft 410, not only when the end tool 100 is not yaw-rotated with respect to the extension shaft 400, as shown in FIGS. 11 to 14, but also when the end tool 100 is yaw-rotated at a certain angle with respect to the extension shaft 400, as shown in FIGS. 7 to 10.

Accordingly, the second roll degree of freedom can be implemented even when the end tool 100 is in a yaw-rotated state.

Meanwhile, the yaw degree of freedom can be implemented by moving the link member 120 forward or backward instead of rotating the link member 120. That is, the end tool 100 can yaw-rotate about the first rotational axis Ax by moving the internal shaft 410 forward or backward along the first longitudinal axis X1.

Specifically, when the internal shaft 410 moves forward toward the distal end 100d, the universal joint bends, and thus the end tool 100 can yaw-rotate at a greater angle. Conversely, when the end tool 100 is in a yaw-rotated state and the internal shaft 410 moves backward toward the proximal end 100p, the universal joint straightens, and thus the end tool 100 can return to a neutral state without yaw rotation.

Meanwhile, the first roll degree of freedom may be implemented through the extension shaft 400. That is, by roll-rotating the extension shaft 400 itself, the entire end tool 100 connected to the extension shaft 400 can rotate together therewith.

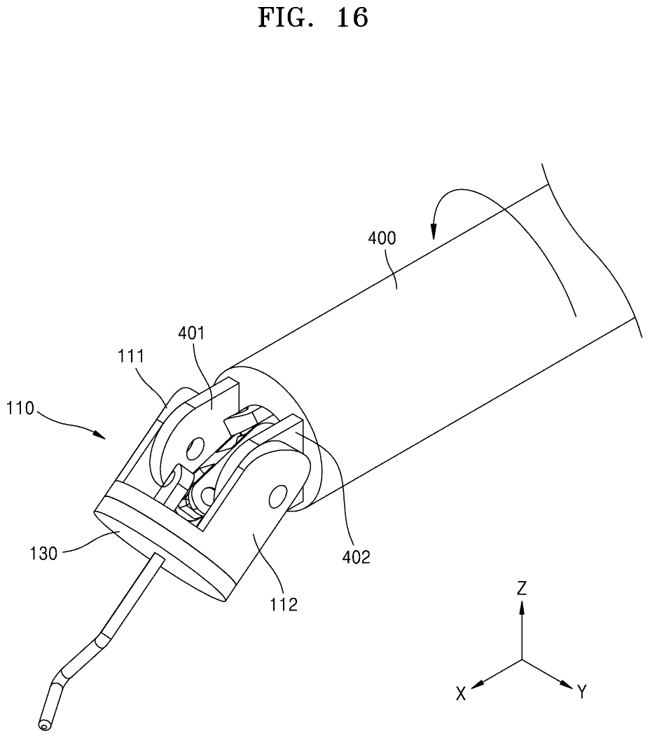

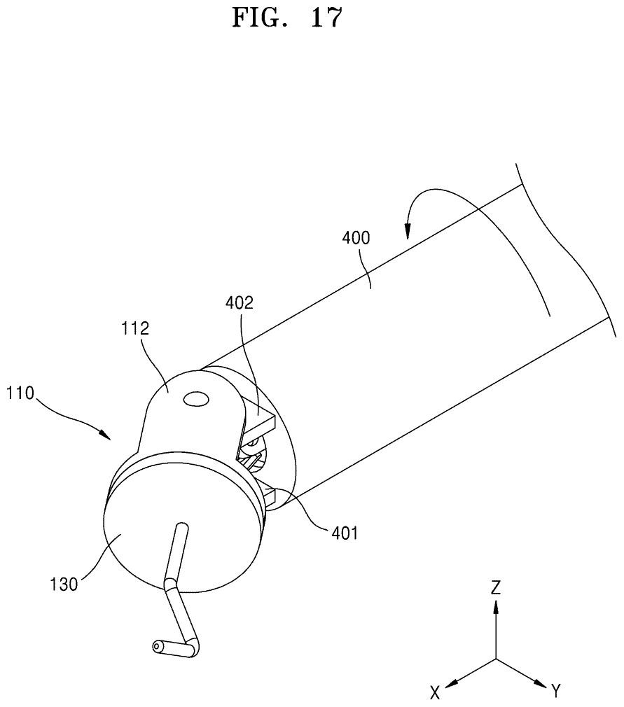

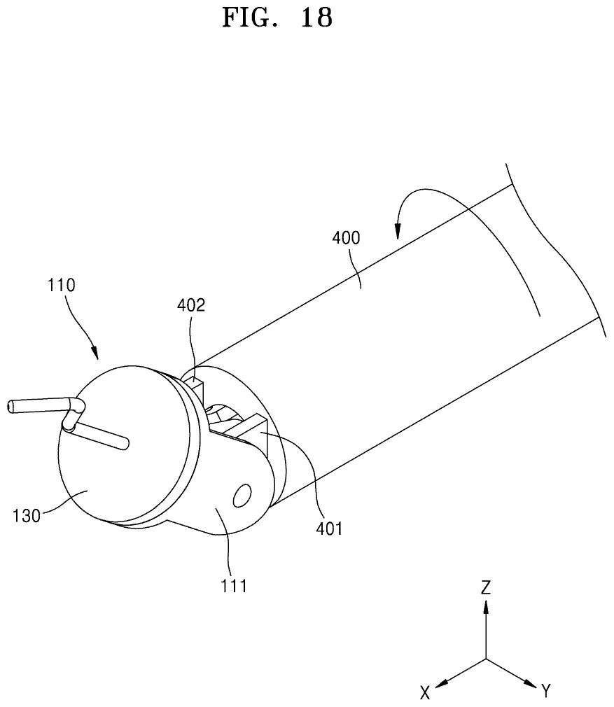

FIG. 15 is a perspective view illustrating a state in which the end tool and shaft assembly 10 of FIG. 5 is yaw-rotated at a certain angle with respect to the first longitudinal axis X1. FIG. 16 is a perspective view illustrating a state in which the end tool and shaft assembly 10 of FIG. 15 is roll-rotated counterclockwise by 90°, FIG. 17 is a perspective view illustrating a state in which the end tool and shaft assembly 10 of FIG. 16 is roll-rotated counterclockwise by 90°, and FIG. 18 is a perspective view illustrating a state in which the end tool and shaft assembly 10 of FIG. 13 is roll-rotated counterclockwise by 90°.

Referring to FIGS. 15 and 18, the end tool and shaft assembly 10 according to an embodiment of the present disclosure enables the end tool 100 to rotate vertically and horizontally by implementing the first roll degree of freedom and the yaw degree of freedom. In other words, the end tool and shaft assembly 10 according to an embodiment of the present disclosure enables the end tool 100 to rotate across the entire range of rotation required in laparoscopic surgery. In addition, the end tool and shaft assembly 10 according to an embodiment of the present disclosure enables the operation-performing portion of the end tool 100 to roll-rotate even when the end tool 100 is in a rotated state by implementing the second roll degree of freedom. As a result, the operation-performing portion can be driven at various angles.

Meanwhile, in implementing the first roll degree of freedom, the internal shaft 410 can rotate together with the extension shaft 400 to induce the rotation of the entire end tool 100 without involving the roll rotation of the end tool plate 130 and the operation-performing portion.

To this end, a driving part for operating the end tool 100 includes a first driving part configured to control the first roll degree of freedom and a yaw driving part configured to control the yaw degree of freedom, and a mechanism for roll-rotating the entire first driving part and yaw driving part may be separately provided.

In other words, the end tool and shaft assembly 10 according to an embodiment of the present disclosure may include the driving part capable of roll-rotating the extension shaft 400 while simultaneously roll-rotating the internal shaft 410.

Hereinafter, an end tool and shaft assembly according to a second embodiment of the present disclosure will be described.

Here, the end tool and shaft assembly according to the second embodiment of the present disclosure is different from the end tool and shaft assembly according to the first embodiment of the present disclosure described above in that a configuration of an end tool is changed. Such a configuration that is changed from that of the first embodiment will be described in detail below.

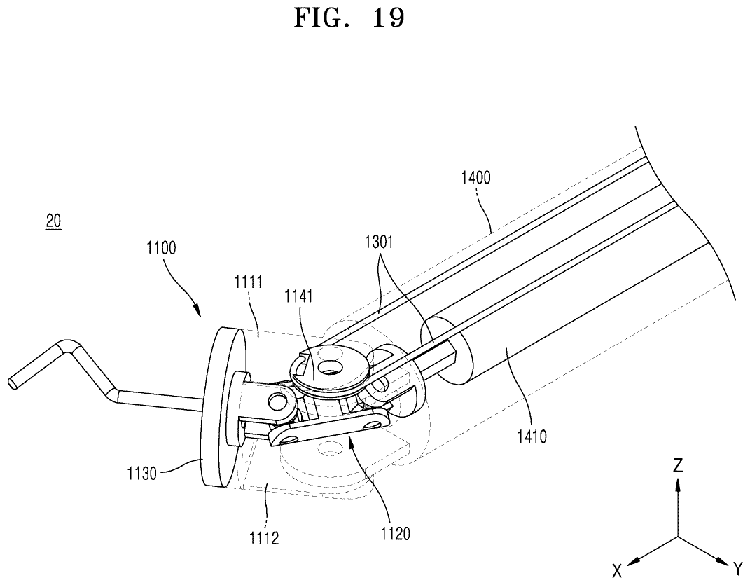

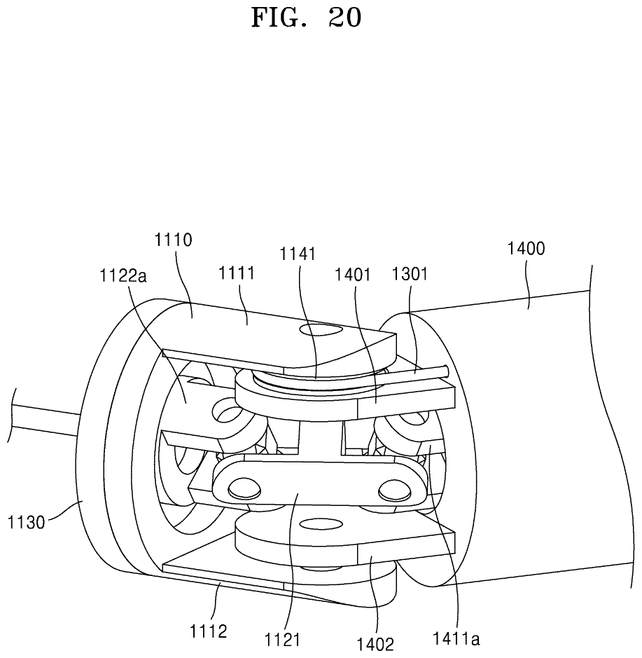

FIG. 19 is a perspective view illustrating the end tool and shaft assembly according to the second embodiment of the present disclosure, and FIG. 20 is a magnified perspective view of the end tool and shaft assembly of FIG. 19 viewed from another angle.

Referring to FIGS. 19 and 20, an end tool and shaft assembly 20 of the second embodiment of the present disclosure may include an end tool 1100, an extension shaft 1400, and an internal shaft 1410. In addition, the end tool 1100 may include a first hub 1110, an end tool plate 1130, and a link member 1120.

Here, the first hub 1110, the end tool plate 1130, the link member 1120, the extension shaft 1400, and the internal shaft 1410 share commonality with the first hub 110, the end tool plate 130, the link member 120, the extension shaft 400, and the internal shaft 410 of the first embodiment in terms of their correspondence. As the common aspects have been described in detail above through the description of the end tool and shaft assembly 10 according to the first embodiment, the following description will focus solely on the distinct differences.

The end tool 1100 of the end tool and shaft assembly 20 according to the second embodiment of the present disclosure may further include a yaw pulley 1141 and a yaw wire 1301.

The yaw pulley 1141 may be disposed in the first hub 1110. Specifically, the yaw pulley 1141 may be disposed between a first coupling portion 1111 and a second coupling portion 1112. Specifically, the yaw pulley 1141 may be disposed between the first coupling portion 1111 and a third coupling portion 1401. In addition, the yaw pulley 1141 may be rotatable about the first rotational axis. In other words, a rotational axis of the yaw pulley 1141 may be identical to a first sub-shaft that axially couples the first coupling portion 1111 to the third coupling portion 1401.

In addition, the yaw pulley 1141 may rotate independently of the third coupling portion 1401 but may rotate integrally with the first coupling portion 1111. Thus, when the yaw pulley 1141 rotates about the first rotational axis, a rotational force is transmitted to the first coupling portion 1111, and the first hub 1110 may rotate together with the yaw pulley 1141.

A groove capable of accommodating a wire may be formed in the yaw pulley 1141.

The yaw wire 1301 is connected to the yaw pulley 1141 and may transmit power to the yaw pulley 1141. Specifically, the yaw wire 1301 may be at least partially wound around the groove of the yaw pulley 1141 and may extend toward the extension shaft 1400. Furthermore, the yaw wire 1301 may be connected to a manipulation part via the extension shaft 1400. Accordingly, through the manipulation of the manipulation part, the yaw wire 1301 may move forward and backward, which causes the yaw pulley 1141 to rotate clockwise or counterclockwise.

By additionally providing the yaw pulley 1141 on the end tool 1100 as described above, the operability of the yaw rotation of the end tool 1100 can be improved.

Specifically, when yaw-rotating the end tool 1100 by the forward and backward movement of the internal shaft 1410, there may be a singularity in a neutral state of the end tool 1100, in which it becomes uncertain whether the end tool 1100 will rotate in a positive (+) direction or a negative (−) direction when the internal shaft 1410 is moved forward toward a distal end.

On the other hand, when yaw-rotating the end tool 1100 through the rotation of the yaw pulley 1141, the end tool 1100 can be manipulated in the desired direction even in its neutral state without yaw rotation. Thus, the operability of the yaw rotation of the end tool 1100 can be improved.

Meanwhile, not only a mechanism of rotating the yaw pulley 1141 but also a mechanism of moving backward and backward the internal shaft 1410 may be simultaneously applied to yaw-rotate the end tool 1100.

Alternatively, only the mechanism of rotating the yaw pulley 1141 may be applied to yaw-rotate the end tool 1100, and the internal shaft 1410 may move forward or backward along the first longitudinal axis as a result of the yaw rotation of the end tool 1100.

In other words, the internal shaft 1410 may passively move forward or backward in response to the yaw rotation of the end tool 1100. At this time, the internal shaft 1410 may transmit only a roll rotational force to the end tool 1100.

However, when the link member 1120 includes a hinge portion, and the hinge portion and a yaw rotation shaft lie in the same plane, the link member 1120 may not move forward or backward even when yaw rotation is driven. Alternatively, even when the link member 1120 includes a double cardan joint, when two yaw rotation shafts are provided in the same plane as two hinge portions, the link member 1120 may not move forward or backward even when yaw rotation is driven.

Hereinafter, an end tool and shaft assembly according to a third embodiment of the present disclosure will be described. Here, the end tool and shaft assembly according to the third embodiment of the present disclosure is different from the end tool and shaft assembly according to the first embodiment of the present disclosure described above in that a configuration of an end tool is changed. Such a configuration that is changed from that of the first embodiment will be described in detail below.

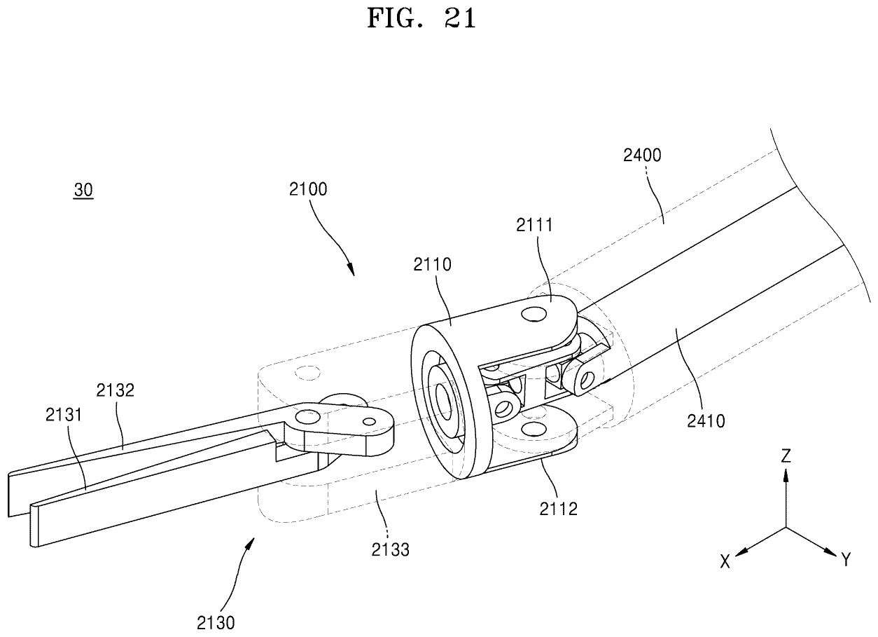

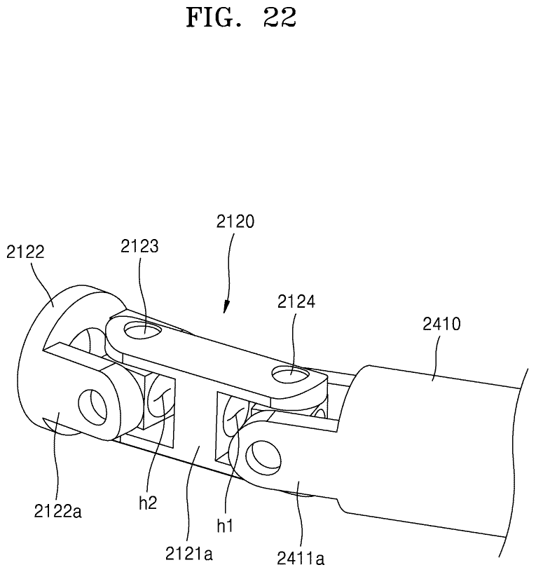

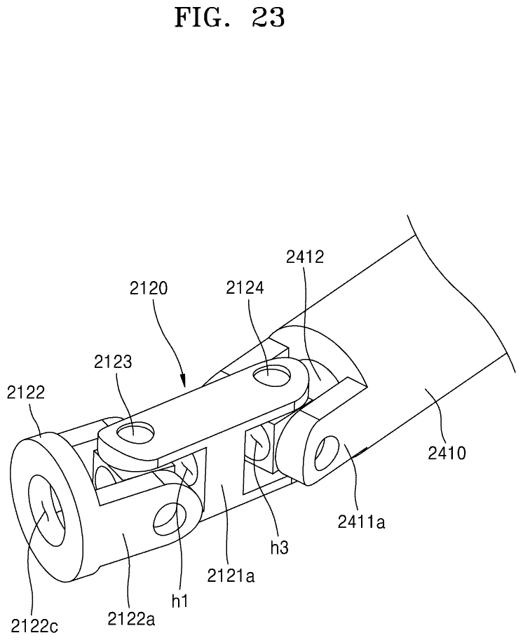

FIG. 21 is a perspective view illustrating the end tool and shaft assembly according to the third embodiment of the present disclosure, and FIGS. 22 and 23 are views for describing a link member and a shaft of the end tool and shaft assembly of FIG. 21.

Referring to FIGS. 21 to 23, an end tool and shaft assembly 30 of the third embodiment of the present disclosure may include an end tool 2100, an extension shaft 2400, and an internal shaft 2410. In addition, the end tool 2100 may include a first hub 2110, an end tool plate 2130, and a link member 2120.

Here, the first hub 2110, the end tool plate 2130, the link member 2120, the extension shaft 2400, and the internal shaft 2410 share commonality with the first hub 110, the end tool plate 130, the link member 120, the extension shaft 400, and the internal shaft 410 of the first embodiment in terms of their correspondence. As the common aspects have been described in detail above through the description of the end tool and shaft assembly 10 according to the first embodiment, the following description will focus solely on the distinct differences.

The end tool 2100 of the end tool and shaft assembly 30 according to the third embodiment of the present disclosure may include a jaw assembly 2130.

Here, the jaw assembly 2130 may be a component including one or more jaws and configured to couple to a distal end of the end tool 2100. For example, the jaw assembly 2130 may be a forceps-shaped surgical tool, such as a dissector.

The jaw assembly 2130 may include a body portion 2133, and one or more jaws may be rotatably coupled to the body portion 2133.

Here, the body portion 2133 may be coupled to the end tool plate. Alternatively, the body portion 2133 may be a component that replaces the end tool plate 130 of the first embodiment described above.

The link member 2120 may connect the internal shaft 2410 to the jaw assembly 2130 and transmit a rotational motion of the internal shaft 2410 to the jaw assembly 2130.

In addition, although not illustrated in the drawings, the end tool 2100 may further include a drive wire (not shown) that is connected to the jaw to cause the jaw to rotate.

In addition, a fixing portion to which the wire may be fixed may be formed on an end portion of the jaw at a proximal end side.

When the drive wire is pulled toward the proximal end, a pair of jaws may engage with each other. Further, although not illustrated in the drawings, an elastic member (not shown) may be provided to exert force in a direction that opens the pair of jaws. Thus, when a force exerted by the drive wire to close and engage each jaw decreases, the jaws may open under the force of the elastic member.

Alternatively, the end tool 2100 may include a separate wire and open each jaw through the operation of the separate wire.

Meanwhile, the link member 2120 may include through holes h1, h2, and h3. The through holes are provided to allow the drive wire to pass therethrough into the link member, and the drive wire may extend through the through holes toward the extension shaft.

Specifically, when the link member 2120 includes a double cardan joint, a first spider, a second spider, and a central body may each include a through hole. In addition, a first yoke and a second yoke may also each include a through hole. In other words, the drive wire may pass sequentially through a through hole 2122c of the first yoke, the through hole h2 of the first spider, the through hole h1 of the central body, the through hole h3 of the second spider, and a through hole 2412 of the second yoke and extend toward the extension shaft.

When the link member 2120 includes a flexible shaft instead of a universal joint, the drive wire may be positioned in an internal space of the corresponding flexible shaft.

Meanwhile, the end tool 2100 may further include a guide tube (not shown). Here, the guide tube may accommodate at least a portion of the drive wire therein, and may be formed to be bendable to a certain degree.

The drive wire may pass through the inside of the guide tube and be connected to the jaw. The drive wire may be configured to move along the guide tube within the guide tube. The guide tube may be made of an elastic plastic or a film.

As described above, the end tool 2100 according to an embodiment of the present disclosure allows the drive wire to move within the guide tube to move the jaw. Accordingly, even when the drive wire moves within the through hole of the link member 2120, damage to the wire may be prevented.

Hereinafter, an end tool and shaft assembly according to a fourth embodiment of the present disclosure will be described. Here, the end tool and shaft assembly according to the fourth embodiment of the present disclosure is different from the end tool and shaft assembly according to the first embodiment of the present disclosure described above in that a configuration of an end tool is changed. Such a configuration that is changed from that of the first embodiment will be described in detail below.

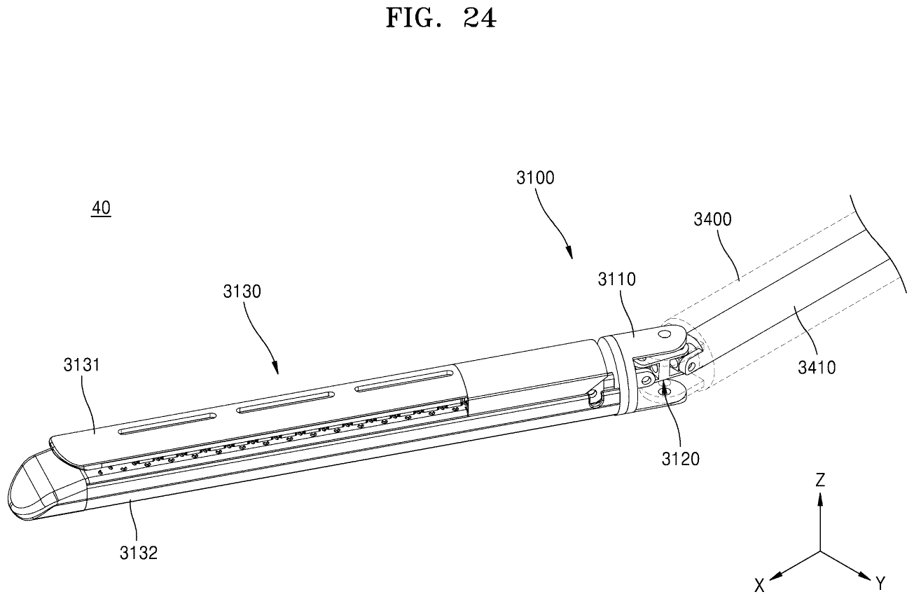

FIG. 24 is a perspective view illustrating the end tool and shaft assembly according to the fourth embodiment of the present disclosure.

Referencing FIG. 24, an end tool and shaft assembly 40 of the fourth embodiment of the present disclosure may include an end tool, an extension shaft, and an internal shaft. In addition, the end tool may include a first hub, an end tool plate, and a link member.

Here, a first hub 3110, an end tool plate (not shown), a link member 3120, an extension shaft 3400, and an internal shaft 3410 share commonality with the first hub 110, the end tool plate 130, the link member 120, the extension shaft 400, and the internal shaft 410 of the first embodiment in terms of their correspondence. As the common aspects have been described in detail above through the description of the end tool and shaft assembly 10 according to the first embodiment, the following description will focus solely on the distinct differences.

An end tool 3100 of the end tool and shaft assembly 40 according to the fourth embodiment of the present disclosure may include a jaw assembly 3130. Here, the jaw assembly 3130 may include a stapler.

That is, a first jaw 3131 may include an anvil (not shown), and a second jaw 3132 may include a staple cartridge accommodation part (not shown).

In addition, the jaw assembly 3130 may include an operation member (not shown) that may move between a proximal end and a distal end in a longitudinal direction. Here, the operation member may push and raise a withdrawal member (not shown) that supports staples of a cartridge to perform a stapling operation.

Here, the operation member may be driven through a wire or a metal strip of a flexible material. At this time, the wire or metal strip may be configured to extend into the interior of the extension shaft through a through hole in the link member, as in the end tool of the third embodiment.

FIG. 25 is a perspective view illustrating a surgical instrument 4000 to which the end tool and shaft assembly according to an embodiment of the present disclosure is applied.

Referring to FIG. 25, the end tool and shaft assembly according to an embodiment of the present disclosure may be connected to a manipulation part 4200.

The manipulation part 4200 may be provided in an interface form that can be directly controlled by a medical doctor, such as a gun shape, a forceps shape, a stick shape, or a lever shape. Here, the manipulation part 4200 is illustrated as being formed in a gun shape, but the concept of the present disclosure is not limited thereto, and various types of manipulation parts capable of being connected to the end tool 4100 and manipulating the end tool 4100 may be possible.

Alternatively, the manipulation part 4200 may be replaced with a surgical robot. In other words, the end tool and shaft assembly may be connected to a drive module of the surgical instrument, and the drive module may be mounted on the surgical robot to enable the manipulation of the end tool 4100.

The manipulation part 4200 may include a first driving part (not shown) configured to drive the first roll degree of freedom, a second driving part (not shown) configured to drive the joint motion degree of freedom of the end tool, and a third driving part (not shown) configured to drive the second roll degree of freedom. In addition, the manipulation part 4200 may be capable of individually driving each degree of freedom.

In addition, the manipulation part 4200 may include a mechanism for roll-rotating the entire second and third driving parts. Specifically, the first driving part may include a mechanism for roll-rotating the entire second and third driving parts.

In other words, the surgical instrument 4000 according to an embodiment of the present disclosure may include a driving part capable of roll-rotating an extension shaft 4400 while simultaneously roll-rotating the internal shaft.

In other words, in implementing the first roll degree of freedom, the surgical instrument 4000 may allow the internal shaft and the link member to rotate together with the extension shaft. Thus, the surgical instrument 4000 according to an embodiment of the present disclosure can roll-rotate the entire end tool 4100 without involving the roll rotation of the end tool plate or the operation-performing portion.

Further, when the end tool 4100 roll-rotates about the second longitudinal axis, a positional relationship between the second longitudinal axis and the manipulation part 4200 may remain unchanged.

In other words, unless the first roll degree of freedom and the joint motion degree of freedom of the surgical instrument 4000 are driven, the positional relationship between the axis of the end tool 4100 and the manipulation part 4200 may remain unchanged when the second roll degree of freedom of the surgical instrument 4000 is driven.

In the surgical instrument according to an embodiment of the present disclosure, one of the yaw and pitch degrees of freedom is removed, and an additional roll degree of freedom, which rotates about the axis of the end tool, is added, thereby implementing the movements required for laparoscopic surgery.

That is, the end tool of the surgical instrument can rotate within a 180° hemisphere or a smaller angle of a spherical cone, while simultaneously rotating about its own axis.

In other words, in the surgical instrument, the end tool needs to be movable vertically and horizontally relative to an extension shaft while also being roll-rotatable about its own axis. For example, surgical forceps consisting of a pair of jaws may perform an opening and closing motion vertically, or may perform a roll rotation to execute the opening and closing motion horizontally. This allows the surgical forceps to grasp body tissues located vertically or horizontally, while also enabling control over whether the forceps grasp the tissues in a vertical or horizontal direction.

Accordingly, in the surgical instrument according to an embodiment of the present disclosure, the size of the joint part of the end tool may be reduced, and a rotation radius of the end tool may be minimized by removing one of yaw or pitch axes. In addition, the surgical instrument of an embodiment of the present disclosure may allow the end tool to move omnidirectionally while reducing wire usage.

According to the present disclosure, a surgical instrument can minimize wire usage while enabling an omnidirectional movement of an end tool.

The present disclosure has been described above in relation to its preferred embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. An end tool and shaft assembly comprising:
an extension shaft defining a first longitudinal axis; and
an end tool connected to one end portion of the extension shaft and configured to perform a joint motion about a first rotational axis perpendicular to the first longitudinal axis,
wherein the end tool defines a second longitudinal axis based on an extension direction of the end tool, and is configured to perform a second roll rotational motion using the second longitudinal axis as a rotational axis and a first roll rotational motion using the first longitudinal axis as a rotational axis.

2. The end tool and shaft assembly of claim 1, wherein the second longitudinal axis is configured to be movable relative to the first longitudinal axis as the end tool performs the joint motion about the first rotational axis.

3. The end tool and shaft assembly of claim 1, wherein the first roll rotational motion and the second roll rotational motion are performed independently of each other.

4. The end tool and shaft assembly of claim 1, further comprising an internal shaft disposed inside the extension shaft and connected to the end tool,
wherein the internal shaft is configured to roll rotate about the first longitudinal axis and is configured to be roll-rotatable independently of the extension shaft.

5. The end tool and shaft assembly of claim 4, wherein the end tool includes:
a first hub connected to the extension shaft configured to be rotatable about the first rotational axis; and
a link member at least partially disposed inside the first hub and configured to connect the internal shaft to the end tool.

6. The end tool and shaft assembly of claim 5, wherein the link member is configured to transmit a translational motion or a rotational motion of the internal shaft to the end tool.

7. The end tool and shaft assembly of claim 6, wherein the end tool is configured to be yaw-rotatable about the first rotational axis as the internal shaft moves forward or backward along the first longitudinal axis.

8. The end tool and shaft assembly of claim 6, wherein the link member is configured to transmit the translational motion or the rotational motion of the internal shaft to the end tool even when the end tool is in a yaw-rotated state.

9. The end tool and shaft assembly of claim 5, wherein the end tool further includes:
a yaw pulley rotatable about the first rotational axis; and
a yaw wire connected to the yaw pulley and configured to transmit a power to the yaw pulley.

10. The end tool and shaft assembly of claim 9,
wherein the yaw pulley is coupled to the first hub, and
wherein the end tool is configured to yaw rotatable in accordance with a rotation of the yaw pulley.

11. The end tool and shaft assembly of claim 10, wherein the internal shaft is configured to move forward or backward along the first longitudinal axis as an end tool yaw-rotates.

12. The end tool and shaft assembly of claim 5,
wherein the end tool further includes a jaw assembly coupled to a distal end of the end tool and including one or more jaws, and
wherein the link member connects the internal shaft to the jaw assembly and is configured to transmit a rotational motion of the internal shaft to the jaw assembly.

13. The end tool and shaft assembly of claim 12,
wherein the end tool further includes a drive wire connected to the one or more jaws and configured to rotate the one or more jaws,
wherein the link member includes a through hole defined therein, and
wherein the drive wire extends toward the extension shaft through the through hole.

14. The end tool and shaft assembly of claim 13,
wherein the end tool further includes a guide tube configured to accommodate at least a portion of the drive wire therein and configured to be bendable to a predetermined degree, and
wherein the drive wire is connected to the one or more jaws through an inside of the guide tube.

15. The end tool and shaft assembly of claim 12, wherein the jaw assembly includes:
a first jaw including a staple cartridge; and
a second jaw including an anvil.

16. An end tool and shaft assembly comprising:
an extension shaft defining a first longitudinal axis; and
an end tool connected to one end portion of the extension shaft,
wherein the end tool and shaft assembly has a first roll degree of freedom defined by the first longitudinal axis, a joint motion degree of freedom of the end tool rotating about a first rotational axis different from the first longitudinal axis, and a second roll degree of freedom defined by a second longitudinal axis based on an extension direction of the end tool, and wherein the first roll degree of freedom, the joint motion degree of freedom, and the second roll degree of freedom are each individually drivable.

17. A surgical instrument comprising:
an extension shaft defining a first longitudinal axis;
an end tool connected to one end portion of the extension shaft and configured to perform a joint motion about a first rotational axis perpendicular to the first longitudinal axis; and
a manipulation part connected to another end portion of the extension shaft and configured to manipulate an operation of the end tool,
wherein the end tool defines a second longitudinal axis based on an extension direction of the end tool, and is configured to perform a second roll rotational motion using the second longitudinal axis as a rotational axis and a first roll rotational motion using the first longitudinal axis as a rotational axis.

18. The surgical instrument of claim 17,
wherein the surgical instrument has a first roll degree of freedom defined by the first longitudinal axis, a joint motion degree of freedom of the end tool rotating about the first rotational axis different from the first longitudinal axis, and a second roll degree of freedom defined by a second longitudinal axis based on the extension direction of the end tool,
wherein the manipulation part includes a first driving part configured to drive the first roll degree of freedom, a second driving part configured to drive the joint motion degree of freedom of the end tool, and a third driving part configured to drive the second roll degree of freedom, and
wherein the first roll degree of freedom, the joint motion degree of freedom, and the second roll degree of freedom are each individually driven.

19. The surgical instrument of claim 18, wherein the first driving part includes a mechanism configured to roll-rotate an entirety of the second driving part and the third driving part.

20. The surgical instrument of claim 17, wherein when the end tool roll-rotates about the second longitudinal axis, a positional relationship between the second longitudinal axis and the manipulation part remains unchanged.
